(19) United States
(12) Patent Application Publication
(10) Pub. No.: US 2006/0154336 A1
(43) Pub. Date: Jul. 13, 2006

(54) Nucleic acid molecules for optimized CAB-2 expression and their use

(75) Inventors: Sujeewa D. Wijesuriya, Walnut Creek, CA (US); Arnold H. Horwitz, San Leandro, CA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/302,782

(22) Filed: Dec. 14, 2005

Related U.S. Application Data
(60) Provisional application No. 60/635,993, filed on Dec. 14, 2004.

(51) Int. Cl. C07H 21/04; C12P 21/06; C07K 14/74

(52) U.S. Cl. 435/69.1; 435/320.1; 435/325; 530/350; 536/23.5

(57) ABSTRACT

The invention provides for nucleic acid molecules encoding a CAB-2 protein and variants thereof. The invention also provides constructs, transformed eukaryotic cells, and methods of producing CAB-2 protein and variants thereof.

### CROSS REFERENCE TO RELATED APPLICATION

This application claims the benefit of U.S. Provisional Application No. 60/635,993, filed Dec. 14, 2004, the entire contents of which are incorporated herein by this reference.

### BACKGROUND OF THE INVENTION

The complement system is a group of proteins in blood plasma which participate in immune and allergic responses. Several protein regulators of the complement system have been identified which primarily regulate the activity of the complement C3/C5 convertases to prevent excessive complement activation and autolytic destruction of host tissues. This class of regulatory proteins has been collectively referred to as the “regulators of complement activation” (RCA) family (see, e.g., Krushkal et al., Mol. Biol. Evol. 17(11):1718-1730 (2000)). MCP (also known as CD46) and DAF (also known as CD55) are important members of the RCA family. Native MCP and DAF are both plasma membrane-associated molecules that contain four short consensus repeat (SCR) motifs, a Serine-Threonine-Proline (STP) rich region, a transmembrane domain, and cytoplasmic tail.

Ko et al. (U.S. Pat. Nos. 5,679,546 and 5,851,528) disclosed the generation and use of novel chimeric genes and proteins which express the biological activities of both MCP and DAF. Ko et al. called these proteins “Complement Activation Blocker” (CAB) proteins, defined as a recombinant chimeric proteins possessing two different complement inhibiting activities, such as those provided by MCP and DAF. Ko et al. further disclosed that CAB molecules are more effective inhibitors of complement activation than the MCP or DAF proteins, individually or in combination. In particular, the CAB-2 molecule had enhanced inhibitory potency for the classical and alternative complement pathways and showed efficacy as a treatment for complement-induced inflammation in an animal model. CAB-2 retains the signal sequence, 4 SCR units, and the first 2 amino acids of the STP region of MCP fused to the 4 SCR units and STP region of DAF.

An important use of recombinant DNA technology is to isolate the sequence encoding a useful protein and express the protein in an exogenous system that allows for the production of adequate quantities of the protein. Accordingly, a common strategy employed within the biotechnology industry is to develop expression systems containing elements proven to yield high-level expression and employ these systems for numerous products. Expression levels, however, can vary from protein to protein, often leading to significant development challenges for poorly expressed proteins. The CAB-2 protein has been expressed in such exogenous systems (U.S. Pat. Nos. 5,679,546 and 5,851,528), but even after some refinement of these systems (U.S. Pat. No. 6,316,253), CAB-2 expression levels have remained inadequate for use in large-scale development and production. The further development of CAB-2 as a pharmaceutical requires improved nucleic acid molecules and methods of expression of CAB-2 that result in higher yields of CAB-2 proteins.

The invention provides such nucleic acid molecules and methods of their use to produce increased amounts of the CAB-2 protein. These and other advantages of the invention, as well as additional inventive features, will be apparent from the description of the invention provided herein.

### BRIEF SUMMARY OF THE INVENTION

The invention provides for a nucleic acid molecule consisting of a mutagenized sequence of SEQ ID NO: 1 which encodes a functional CAB-2 protein. The mutagenized sequence of SEQ ID NO: 1 results from selecting changes from the group consisting of changing the A at nucleotide 501 to G, changing the A at nucleotide 504 to C or T, changing the A at nucleotide 1170 to G, changing the T at nucleotide 1173 to A, C or G, changing the T at nucleotide 1524 to C, changing the A at nucleotide 1527 to G, changing the T at nucleotide 1800 to C, changing the A at nucleotide 1803 to G, and combinations thereof. The invention includes combinations of these nucleotide changes. The inventive nucleic acid molecule is exemplified (without limitation) by the nucleic acid molecule of SEQ ID NO: 2.

In one embodiment, the invention provides a CAB-2 nucleic acid molecule having the nucleotide sequence of the DNA insert of the plasmid deposited with ATCC as Accession Number ______. In another embodiment, the invention provides nucleic acid molecules that are sufficiently or substantially identical (e.g., a variant) to the nucleotide sequence of the DNA insert of the plasmid deposited with ATCC as Accession Number ______. In another embodiment, the invention provides nucleic acid molecules variants of the DNA insert of the plasmid deposited with ATCC as Accession Number ______ which encode a CAB-2 polypeptide which is sufficiently or substantially identical (e.g. a variant with a conservative amino acid change) to SEQ ID NO:3. In still another embodiment, the invention provides a nucleic acid molecule which hybridizes under stringent hybridization conditions to a nucleic acid molecule comprising the nucleotide sequence of the DNA insert of the plasmid deposited with ATCC as Accession Number ______, wherein the nucleic acid encodes a full length CAB-2 protein or an active fragment thereof.

The invention also provides a nucleic acid molecule encoding a protein of the amino acid sequence SEQ ID NO: 3, wherein expression of the nucleic acid molecule produces protein of SEQ ID NO: 3 at a level of at least 60%, preferably at least 70%, more preferably at least 80% that of a nucleic acid molecule consisting of the sequence of SEQ ID NO: 2 or the sequence of the DNA insert of the plasmid deposited with ATCC as Accession Number ______ when transformed into eukaryotic cells, e.g. CHO-K1 cells, and expressed under identical conditions.

In other embodiments, the invention provides a CAB-2 polypeptide encoded by the DNA insert of the plasmid deposited with ATCC as Accession Number ______; an amino acid sequence that is sufficiently or substantially identical to the amino acid sequence encoded by the cDNA insert of the plasmid deposited with ATCC as Accession Number ______; or an amino acid sequence encoded by a nucleic acid molecule having a nucleotide sequence which hybridizes under stringent hybridization conditions to a nucleic acid molecule comprising the nucleotide sequence of the insert of the plasmid deposited with ATCC as Accession Number ______, wherein the nucleic acid encodes a full length CAB-2 protein or an active fragment thereof.

The invention further provides for constructs comprising control elements operatively linked to the inventive nucleic acid molecules for expression of a protein of SEQ ID NO: 3 selected from the group consisting of (a) the change of a leucine, isoleucine, valine, methionine, phenylalanine, serine, cysteine, or threonine to a leucine, isoleucine, valine, methionine, phenylalanine, serine, cysteine, or threonine, (b) the change of a glycine, alanine, valine, serine, cysteine, or threonine to a glycine, alanine, valine, serine, cysteine, or threonine, (c) the change of a phenylalanine, tyrosine, or tryptophan to a phenylalanine, tyrosine, or tryptophan, (d) the change of a glutamic acid, aspartic acid, glutamine, or asparagine to a glutamic acid, aspartic acid, glutamine, or asparagine (e) the change of a histidine, lysine, or arginine to a histidine, lysine, or arginine, and (f) the change of a serine, threonine, or cysteine to a serine, threonine, or cysteine, and combinations thereof, wherein expression of the nucleic acid molecule produces protein of mutagenized protein sequence derived from the sequence of SEQ ID NO: 3 at a level of at least 60%, preferably at least 70%, more preferably at least 80% that of a nucleic acid molecule consisting of the sequence of SEQ ID NO: 2 when transformed into CHO-K1 cells under identical conditions.

Moreover, the invention provides for a eukaryotic cell transformed with any of the inventive nucleic acid molecules described herein, including where the cell is a CHO-K1 cell. The invention also provides a method of making CAB-2 protein comprising providing cells transformed with an inventive nucleic acid molecule and expressing CAB-2 protein from the cells.

### BRIEF DESCRIPTION OF THE DRAWINGS

FIG. 1 depicts the nucleic acid and related-amino acid sequences of the invention (FIG. 1A, SEQ ID NO: 1; FIG. 1B, SEQ ID NO: 2; FIGS. 1C-1 and 1C-2, SEQ ID NO: 3; FIG. 1D, SEQ ID NO: 4).

FIG. 2 is a schematic representation of a conventional CAB-2 nucleic acid molecule.

FIG. 3 is a graph that shows CAB-2 protein expression from CHO-K1 cells transformed with conventional and inventive (mutated) nucleic acids encoding CAB-2.

### DETAILED DESCRIPTION OF THE INVENTION

The invention relates to CAB-2-encoding nucleic acid molecules which can produce primarily full length transcripts when expressed in a host cell. Preferably, the full length transcript for CAB-2 is at least about 1.84 kilobases, at least 2.0 kilobases, or at least 2.3 kilobases, depending on the choice of primer used to identify the transcript. The invention relates to mutations of the CAB-2-encoding SEQ ID NO:1 wherein the mutations result in expression of at least 60%, 70%, 80%, preferably at least 90%, more preferably at least 95% full length transcripts when expressed in a eukaryotic cell. Mutations within the invention remove at least one polyadenylation signal site, preferably at least two polyadenylation signal sites. Such polyadenylation sites, if left intact, can be used by host cells to prematurely terminate the CAB-2 transcript so it is less than 2.3 kilobases. One polyadenylation site can be removed from the MCP portion of CAB-2, e.g. by a mutation of at least one base, preferably two bases at about bases 501 to 506 of SEQ ID NO:1. Another polyadenylation site can be removed from the DAF portion of CAB-2, e.g., by a mutation of at least one base, preferably two bases at about bases 1522 to 1527 of SEQ ID NO:1.

A further embodiment of the invention is to remove a splice site which also induces a transcript of less than full length CAB-2 mRNA. The splice site can be canonical or non-canonical. A non-canonical splice site within the invention can splice a truncated portion of CAB-2 to the 3′UTR, resulting in a transcript without sequences encoding a portion of DAF. For example, at least one base, preferably two bases in a donor splice site can be mutated at about bases 1170 to 1173 of SEQ ID NO:1.

Preferably, two polyadenylation signals and one splice site are mutated and inactive in the inventive nucleic acid molecules. Preferably, mutation of one or more of these sites preserves the protein sequence of CAB-2 (SEQ ID NO:3), e.g., retains amino acid residues 167 to 167, 390 to 392, and/or 508 to 509, respectively, of SEQ ID NO:3, although a conservative amino acid change can be made to retain CAB-2 activity after mutating the polyadenylation site or splice site. Preferred embodiments of a mutated nucleic acid are a nucleic acid consisting of the nucleotide sequence of SEQ ID NO:2 or the nucleotide sequence of the DNA insert of the plasmid deposited with ATCC as Accession Number ______. Additional polyadenylation signal sites which can be mutated in a CAB-2-encoding nucleic acid can be at about bases 1665 to 1670 and/or at about bases 1798 to 1803 of SEQ ID NO:1, preferably mutated in a way which preserves amino acid residues 555 to 557 and/or 600 to 601, respectively, of SEQ ID NO:3.

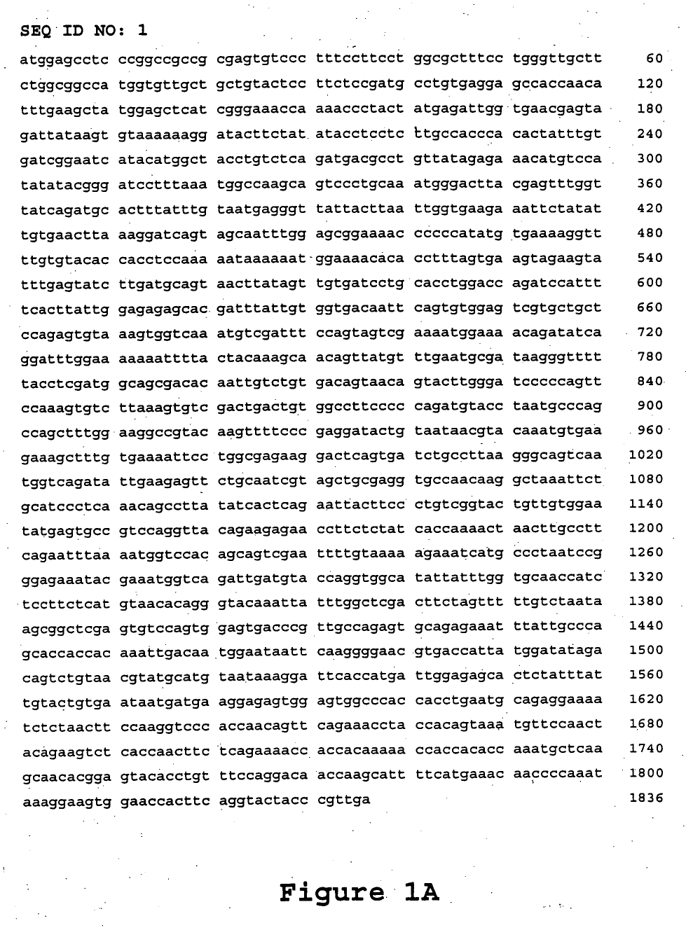

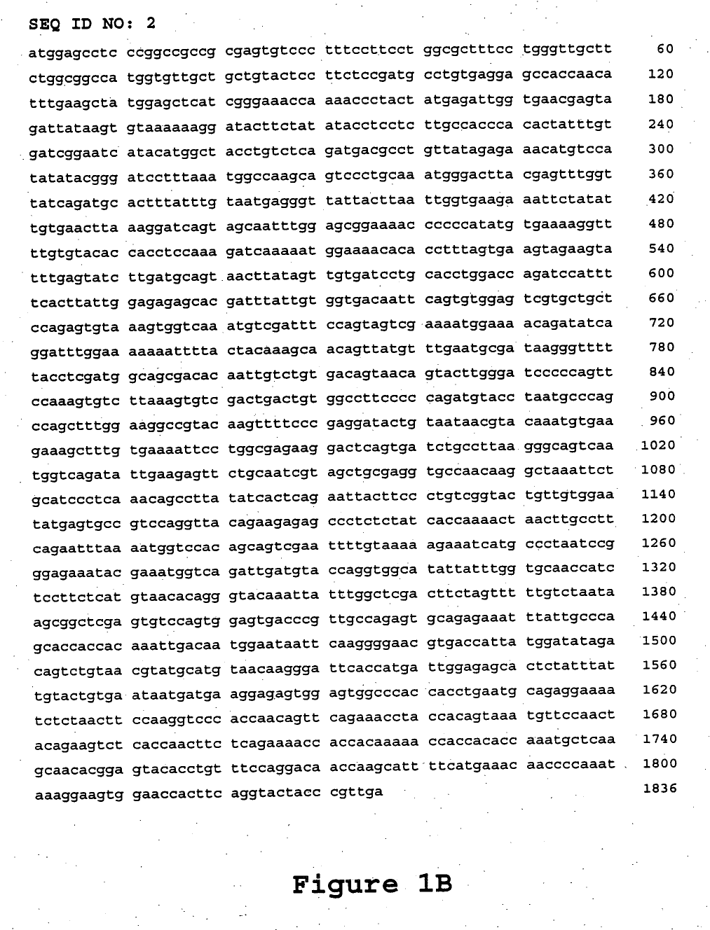

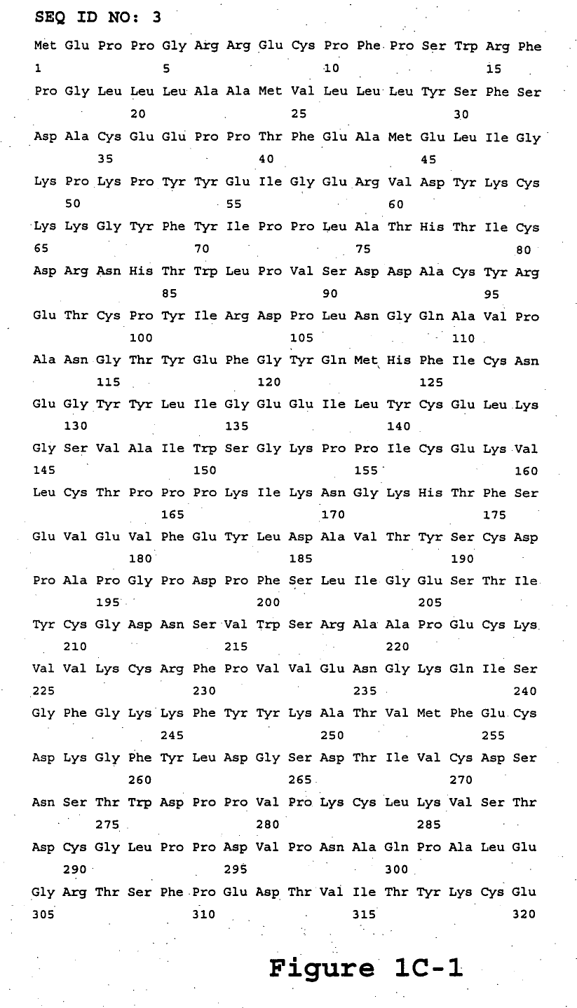

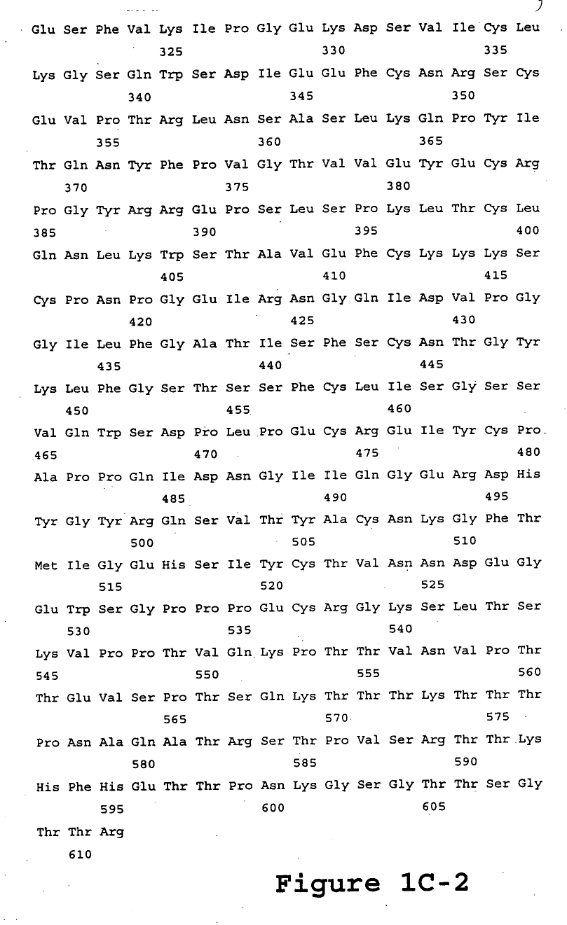

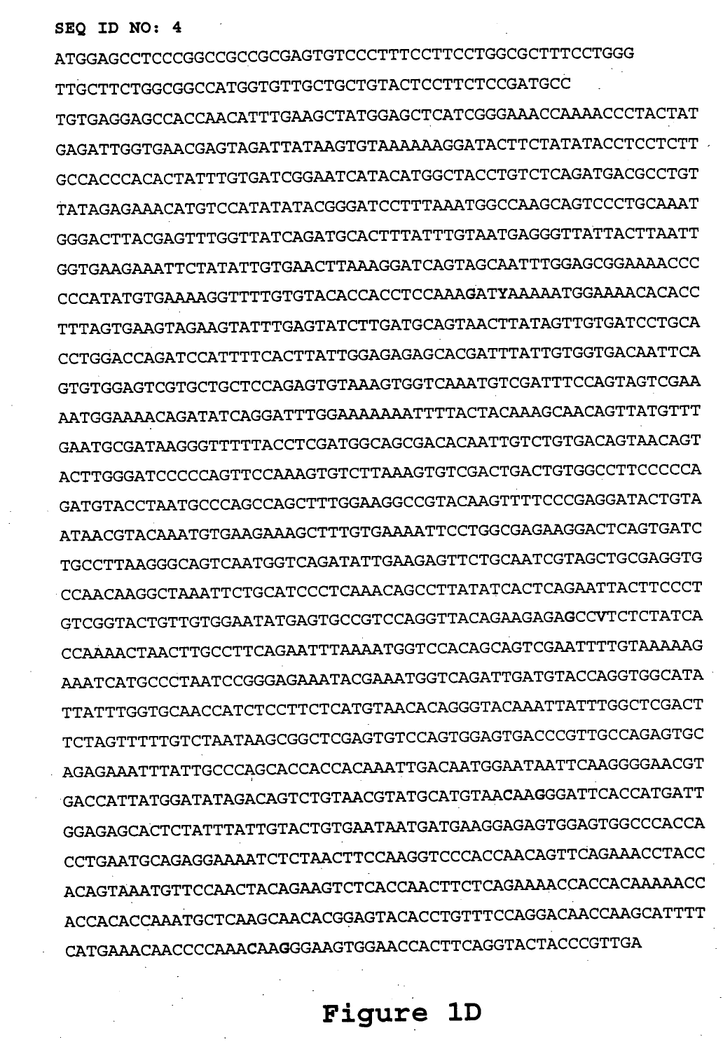

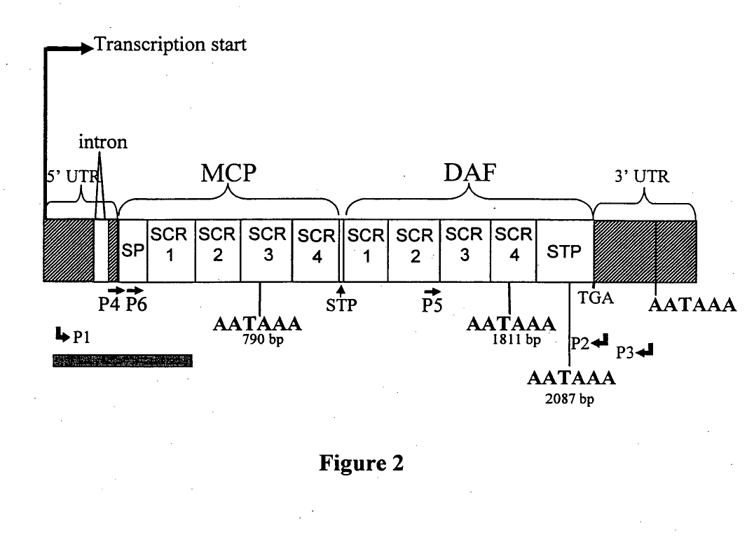

The invention provides for a nucleic acid molecule consisting of a mutagenized sequence of SEQ ID NO: 1 which encodes a functional CAB-2 protein. FIG. 1 depicts the nucleic acid and related-amino acid sequences of the invention (FIG. 1A, SEQ ID NO: 1 DNA sequence; FIG. 1B, SEQ ID NO: 2 DNA sequence; FIGS. 1C-1 and 1C-2, SEQ ID NO: 3 amino acid sequence; FIG. 1D, SEQ ID NO: 4 DNA sequence; SEQ ID NOs:1 and 3 were disclosed in FIGS. 1A-1C of U.S. Pat. No. 6,316,253, incorporated herein by reference). The mutagenized sequence results from selecting changes from the group consisting of changing the A at nucleotide 501 to G, changing the A at nucleotide 504 to C, changing the A at nucleotide 504 to T, changing the A at nucleotide 1170 to G, changing the T at nucleotide 1173 to C, changing the T at nucleotide 1173 to A, changing the T at nucleotide 1173 to G, changing the T at nucleotide 1524 to C, changing the A at nucleotide 1527 to G, changing the T at nucleotide 1800 to C, and changing the A at nucleotide 1803 to G, and combinations thereof. Furthermore, the inventive nucleic acid molecule is exemplified (without limitation to) by the nucleic acid molecule of SEQ ID NO: 2 (FIG. 1B). These mutations and their positions are depicted in FIG. 1D which discloses SEQ ID NO: 4. In FIG. 1D, “Y” represents a pyridmidine nucleotide and “V” represents any nucleotide expect thymine or uracil. While not desiring to be bound by any theory of how the invention works, it is noted that the changes at nucleotides 501, 504, 1524, 1527, 1800, and 1803 can eliminate cryptic polyadenylation sites while the changes at nucleotides 1170 and 1173 can eliminate a cryptic splice donor site.

The nucleic acid molecule desirably has at least two mutations in the nucleic acid sequence of SEQ ID NO: 1, wherein (a) at least one mutation is selected from the group consisting of A at nucleotide 501 to G, changing the A at nucleotide 504 to C, changing the A at nucleotide 504 to T, changing the T at nucleotide 1524 to C, changing the A at nucleotide 1527 to G, changing the T at nucleotide 1800 to C, and changing the A at nucleotide 1803 to G, and (b) at least one mutation is selected from the group consisting of changing the A at nucleotide 1170 to G, changing the T at nucleotide 1173 to C, changing the T at nucleotide 1173 to A, and changing the T at nucleotide 1173 to G.

A plasmid containing a nucleic acid with a mutant sequence encoding a CAB-2 protein, named pING1736A1A2SD, was deposited with American Type Culture Collection (ATCC), 10801 University Boulevard, Manassas, Va. 20110-2209, on Dec. 8, 2005 and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

In addition, the invention includes a nucleic acid molecule that hybridizes with any of the mutagenized nucleic acid molecules of SEQ ID NO: 1 disclosed herein under low stringency conditions or, preferably, under moderate stringency conditions or, most preferably, under high stringency conditions, wherein expression of the nucleic acid molecule produces a protein of SEQ ID NO: 3 at a level of at least 60%, preferably at least 70%, more preferably at least 80% that of a nucleic acid molecule consisting of the sequence of SEQ ID NO: 2 or the sequence of the DNA insert of the plasmid deposited with ATCC as Accession Number ______ when transformed into CHO-K1 cells under identical conditions.

“High stringency conditions” preferably allow for from about 25% to about 5% mismatch, more preferably from about 15% to about 5% mismatch, and most preferably from about 10% to about 5% mismatch of the nucleic acid sequence. “Moderately stringent conditions” preferably allow for from about 40% to about 15% mismatch, more preferably from about 30% to about 15% mismatch, and most preferably from about 20% to about 15% mismatch of the nucleic acid sequence. “Low stringency conditions” preferably allow for from about 60% to about 35% mismatch, more preferably from about 50% to about 35% mismatch, and most preferably from about 40% to about 35% mismatch of the nucleic acid sequence.

The absence of non-specific binding may be tested by the use of a second target which lacks even a partial degree of complementarity (e.g., less than about 30% identity); in the absence of non-specific binding the probe will not hybridize to the second non-complementary target. There may be partial homology or complete homology (i.e., identity). A partially complementary sequence is one that at least partially inhibits a completely complementary sequence from hybridizing to a target nucleic acid. The inhibition of hybridization of the completely complementary sequence to the target sequence may be examined using a hybridization assay (Southern or Northern blot, solution hybridization and the like) under conditions of low stringency. A “substantially homologous” sequence or probe will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The term “homology” refers to a degree of complementarity.

Exemplary moderate stringency conditions include overnight incubation at 37° C. in a solution comprising 20% formamide, 5×SSC (150 mM NaCl and 15 mM trisodium citrate), 50 mM sodium phosphate (pH 7.6), 5× Denhardt's solution, 10% dextran sulfate, and 20 mg/ml denatured sheared salmon sperm DNA, followed by washing in 1×SSC at about 37-50° C., or substantially similar conditions, e.g., the moderately stringent conditions described in Sambrook et al., MOLECULAR CLONING: A LABORATORY MANUAL, Cold Spring Harbor Laboratory Press, New York (1989). High stringency conditions are conditions that, for example (1) use low ionic strength and high temperature for washing, such as with a composition comprising 0.015 M sodium chloride and 0.0015 M sodium citrate, and 0.1% (w/v) sodium dodecyl sulfate (SDS) at 50° C., (2) employ a denaturing agent during hybridization, such as a composition comprising formamide, for example, 50% (v/v) formamide with 0.1% (w/v) bovine serum albumin (BSA), 0.1% Ficoll, 0.1% polyvinylpyrrolidone (PVP), and 50 mM sodium phosphate buffer at pH 7.5 with 750 mM sodium chloride and 75 mM sodium citrate at 42° C., or (3) employ a composition comprising 50% formamide, 5×SSC (0.75 M NaCl and 0.075 M sodium citrate), 50 mM sodium phosphate (pH 6.8), 0.1% sodium pyrophosphate, 5× Denhardt's solution, and sonicated salmon sperm DNA (50 μg/ml), 0.1% SDS, and 10% dextran sulfate at 42° C., with washes at (i) 42° C. in 0.2×SSC, (ii) at 55° C. in 50% formamide, and (iii) at 55° C. in 0.1×SSC (preferably in combination with EDTA).

The invention provides nucleic acid molecules which are at least 70%, preferably at least 80%, more preferably at least 90%, even more preferably at least 95%, even more preferably at least 96%, even more preferably at least 97%, most preferably at least 98% homologous to any of the mutagenized nucleic acid molecules derived from SEQ ID NO: 1 disclosed herein, wherein when the nucleic acid molecule is transformed into eukaryotic cells it expresses a protein of SEQ ID NO: 3 at a level of at least 60%, preferably at least 70%, more preferably at least 80% that of CHO-K1 cells transformed with the nucleic acid of SEQ ID NO: 2. Protein concentration can be measured using ELISA and Reverse Phase HPLC (RP-HPLC). ELISA plates are pre-coated with a murine monoclonal IgG₁ against MCP. After blocking with PBS containing 1% BSA and 0.5% Tween 20, the plates are incubated with cell culture supernatant. This is followed by a two-step incubation with a polyclonal rabbit anti-CAB-2 antibody followed by a mouse anti-rabbit antibody conjugated to horse radish peroxidase. The plates are developed using a suitable detection system, such as, but without limitation to, the TMB detection system (KPL, Gaithersburg Md.) and read on a Biotek ELISA plate reader at 450 nm. To determine CAB-2 protein concentration with the aid of RP-HPLC, culture supernatants from cultures are passed through 0.2 micron filters and analyzed using a Poroshell 300SB-C18 column of 2.1×75 mm (Agilent Technologies, Palo Alto, Calif.) with a gradient elution. Protein titer is then determined by interpolation of sample peak area with a five point calibration curve ranging from 10 μg/mL to 500 μg/mL.

The invention provides a nucleic acid molecule encoding a protein with the amino acid sequence of SEQ ID NO: 3 at a level of at least 60%, preferably at least 70%, more preferably at least 80% that of CHO-K1 cells transformed with a nucleic acid molecule consisting of the sequence of SEQ ID NO: 2 or the sequence of the DNA insert of the plasmid deposited with ATCC as Accession Number ______. Furthermore, the invention provides nucleic acid molecules encoding proteins with conservative changes of SEQ ID NO: 3. The term “conservative change” refers to the replacement (or substitution) of an amino acid with a naturally or non-naturally occurring amino acid having similar steric properties. Where the side-chain of the amino acid to be replaced is either polar or hydrophobic, the conservative change should be with a naturally or non-naturally occurring amino acid that is also polar or hydrophobic (in addition to having the same steric properties as the side-chain of the replaced amino acid). When the native amino acid to be replaced is charged, the conservative change can be with a naturally or non-naturally occurring amino acid that is charged, or with a non-charged (polar, hydrophobic) amino acid that has the same steric properties as the side-chains of the replaced amino acid. For example, the replacement of arginine by glutamine, aspartate by asparagine, or glutamate by glutamine is considered to be a conservative change.

In order to further exemplify what is meant by conservative change, Groups A-F are listed below. The replacement of one member of the following groups by another member of the same group is considered to be a conservative change.

Group A includes leucine, isoleucine, valine, methionine, phenylalanine, serine, cysteine, and threonine.

Group B includes glycine, alanine, valine, serine, cysteine, and threonine.

Group C includes phenylalanine, tyrosine, and tryptophan.

Group D includes glutamic acid and aspartic acid.

Group E includes histidine, lysine, and arginine.

Group F includes serine, threonine, and cysteine.

The nucleic acid molecule can encode a mutagenized protein of the amino acid sequence of SEQ ID NO: 3 with a combination of two or more of the conservative amino acid changes described herein. Furthermore, the nucleic acid molecule desirably provides for the expression of a protein of SEQ ID NO: 3 or a variant thereof with one or more conservative amino acid changes at a level of at least 60%, preferably at least 70%, more preferably at least 80% that of the protein of SEQ ID NO: 3 produced when the nucleic acid molecule consisting of the sequence of SEQ ID NO: 2 or the sequence of the DNA insert of the plasmid deposited with ATCC as Accession Number ______ is transformed into CHO-K1 cells under identical conditions.

Mutagenesis can be undertaken by any of several methods known in the art. Generally, mutagenesis can be accomplished by cloning the nucleic acid sequence into a plasmid or some other vector for ease of manipulation of the sequence. Then, a unique restriction site at which further nucleic acids can be added into the nucleic acid sequence is identified or inserted into the nucleic acid sequence. A double-stranded synthetic oligonucleotide generally is created from overlapping synthetic single-stranded sense and antisense oligonucleotides such that the double-stranded oligonucleotide incorporates the restriction sites flanking the target sequence and, for instance, can be used to incorporate replacement DNA. The plasmid or other vector is cleaved with the restriction enzyme, and the oligonucleotide sequence having compatible cohesive ends is ligated into the plasmid or other vector to replace the original DNA.

Other means of in vitro site-directed mutagenesis are known to those skilled in the art, and can be accomplished (in particular, using an overlap-extension polymerase chain reaction (PCR). For example, without limitation, changes in SEQ ID NO: 1 to inactivate the two functional, internal AATAAA sites and the splice donor site can be made by using inverse PCR and DpnI digestion (Parikh & Guengerich, Biotechniques 24:428-431 (1998)). Complementary primers overlapping the site of change can be used to PCR amplify the whole plasmid in a mixture containing 500 mM dNTPs, 2 units of Pfu polymerase, 250 ng each of sense and antisense primers, and 200 ng of CAB-2 plasmid DNA. The PCR desirably involves 18 cycles with an extension time of 2.5 minutes for each Kb of DNA. The PCR products can be treated with DpnI (which only digests the adenine-methylated plasmid DNA) and transformed into Escherichia coli DH5α cells. Transformants can be screened by restriction enzyme digestion for incorporation of the changes, which then can be confirmed by DNA sequence analysis.

The nucleic acid fragment encoding the mutagenized sequence can be isolated, e.g., by PCR amplification using 5′ and 3′ primers, preferably ones that terminate in further unique restriction sites, that flank the mutated nucleotide. Use of primers in this fashion results in an amplified sequence that is flanked by the unique restriction sites. The unique restriction sites can be used for further convenient subcloning of the fragment.

The invention further provides nucleic acid constructs comprising control elements and a mutagenized nucleic acid molecule described herein operatively linked to the control elements (e.g., a suitable promoter) for expression of protein of SEQ ID NO: 3 or a protein herein described with conservative amino acid changes in SEQ ID NO: 3. Protein expression is dependent on the level of RNA transcription, which is in turn regulated by DNA signals. Similarly, translation of mRNA requires, at the very least, an AUG initiation codon, which is usually located within 10 to 100 nucleotides of the 5′ end of the message. Sequences flanking the AUG initiator codon have been shown to influence its recognition by eukaryotic ribosomes, with conformity to a perfect Kozak consensus sequence resulting in optimal translation (see, e.g., Kozak, J. Molec. Biol. 196: 947-950 (1987)). Also, successful expression of an exogenous nucleic acid in a cell can require post-translational modification of a resultant protein.

The nucleic acid molecules described herein preferably comprise a coding region operatively linked to a suitable promoter, which promoter is preferably functional in eukaryotic cells. Viral promoters, such as, without limitation, the RSV promoter, the immediate early cytomegalovirus (CMV) promoter (Boshart et al. (1985) Cell 41:521-530) and the adenovirus major late promoter can be used in the invention. Suitable non-viral promoters include, but are not limited to, the phosphoglycerokinase (PGK) promoter and the elongation factor 1α promoter. Non-viral promoters are desirably human promoters. Additional suitable genetic elements, many of which are known in the art, also can be ligated to, attached to, or inserted into the inventive nucleic acid and constructs to provide additional functions, level of expression, or pattern of expression. The native promoters for expression of the RCA family genes also can be used, in which event they are preferably not used in the chromosome naturally encoding them unless modified by a process that substantially changes that chromosome. Such substantially changed chromosomes can include chromosomes transfected and altered by a retroviral vector or similar process. Alternatively, such substantially changed chromosomes can comprise an artificial chromosome such as a HAC, YAC, or BAC.

In addition, the nucleic acid molecules described herein may be operatively linked to enhancers to facilitate transcription. Enhancers are cis-acting elements of DNA that stimulate the transcription of adjacent genes. Examples of enhancers which confer a high level of transcription on linked genes in a number of different cell types from many species include, without limitation, the enhancers from SV40 and the RSV-LTR. Such enhancers can be combined with other enhancers which have cell type-specific effects, or any enhancer may be used alone.

To optimize protein production the inventive nucleic acid molecule can further comprise a polyadenylation site following the coding region of the nucleic acid molecule. Also, preferably all the proper transcription signals (and translation signals, where appropriate) will be correctly arranged such that the exogenous nucleic acid will be properly expressed in the cells into which it is introduced. If desired, the exogenous nucleic acid also can incorporate splice sites (i.e., splice acceptor and splice donor sites) to facilitate mRNA production while maintaining an inframe, full length transcript. Moreover, the inventive nucleic acid molecules can further comprise the appropriate sequences for processing, secretion, intracellular localization, and the like.

The nucleic acid molecules can be inserted into any suitable vector. Suitable vectors include, without limitation, viral vectors. Suitable viral vectors include, without limitation, retroviral vectors, alphaviral, vaccinial, adenoviral, adenoassociated viral, herpes viral, and fowl pox viral vectors. The vectors preferably have a native or engineered capacity to transform eukaryotic cells, preferably CHO-K1 cells. Additionally, the vectors useful in the context of the invention can be “naked” nucleic acid vectors (i.e., vectors having little or no proteins, sugars, and/or lipids encapsulating them) such as plasmids or episomes, or the vectors can be complexed with other molecules. Other molecules that can be suitably combined with the inventive nucleic acids include without limitation viral coats, cationic lipids, liposomes, polyamines, gold particles, and targeting moieties such as ligands, receptors, or antibodies that target cellular molecules.

As used herein, the terms “transform” or “transfect” refer to the introduction of nucleic acids, e.g. a plasmid, into a host cell, e.g. prokaryotic cell or eukaryotic cell.

The nucleic acid molecules described herein can be transformed into any suitable cell, typically a eukaryotic cell, such as CHO, HEK293, or BHK, preferably a CHO cell, most preferably CHO-K1 cell (ATCC, Manassas, Va.), desirably resulting in the expression of a protein of SEQ ID NO: 3 or a variant thereof. The cell can be cultured to provide for the expression of the nucleic acid molecule and, therefore, the production of the CAB-2 protein having the amino acid sequence of SEQ ID NO: 3 or a variant thereof.

Therefore, the invention provides for a cell transformed or transfected with an inventive nucleic acid molecule described herein. Means of transforming, or transfecting, cells with exogenous DNA molecules are well known in the art. For example, without limitation, a DNA molecule is introduced into a cell using standard transformation or transfection techniques well known in the art such as calcium-phosphate or DEAE-dextran-mediated transfection, protoblast fusion, electroporation, liposomes and direct microinjection (see, Sambrook et al., MOLECULAR CLONING: A LABORATORY MANUAL, Cold Spring Harbor Laboratory Press, New York (1989)).

A widely used method for transformation is transfection mediated by either calcium phosphate or DEAE-dextran. Depending on the cell type, up to 20% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that carry integrated copies of the foreign DNA.

Another example of a transformation method is the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells, and the plasmid DNA is transferred to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA randomly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of clones genes and for establishment of cell lines that carry integrated copies of the gene of interest.

Liposome transformation involves encapsulation of DNA and RNA within liposomes, followed by fusion of the liposomes with the cell membrane. In addition, DNA that is coated with a synthetic cationic lipid can be introduced into cells by fusion. Alternatively, linear and/or branched polyethylenimine (PEI) can be used in transfection.

Direct microinjection of a DNA molecule into nuclei has the advantage of not exposing the DNA molecule to cellular compartments such as low-pH endosomes. Microinjection is, therefore, used primarily as a method to establish lines of cells that carry integrated copies of the DNA of interest.

Such techniques can be used for both stable and transient tranformation of eukaryotic cells. The isolation of stably transformed cells requires the introduction of a selectable marker in conjunction with the transformation with the gene of interest. Such selectable markers include genes which confer resistance to neomycin as well as the HPRT gene in HPRT-negative cells. Selection can require prolonged culture in selection media, at least for about 2-7 days, preferable for at least about 1-5 weeks (see, e.g., Pollard et al., METHODS IN MOLECULAR BIOLOGY VOL. 5: ANIMAL CELL CULTURE, Humana Press (1990); Masters, ANIMAL CELL CULTURE—A PRACTICAL APPROACH, 3d ed., Oxford University Press (2000)).

In a preferred embodiment, the exogenous DNA molecule is a linearized vector containing the mutated CAB-2 nucleic acid described herein under control of the CMV promoter. The transfection method preferably employs linear PEI. The transfection method or the vector can be adjusted to increase the number of copies of the CAB-2 encoding nucleic acid introduced and/or expressed in the host cell. Examples of such vectors and methods can be found in U.S. patent application Ser. No. 10/404,724, published as U.S. patent application publication No. 2003/0203447, incorporated herein by reference.

Another aspect the invention is a method of making CAB-2 protein or a variant thereof as described herein. The method comprises providing cells transformed with any of the inventive nucleic acid molecules described herein, expressing the nucleic acid molecules, and thereby producing CAB-2 protein or a variant thereof from the transformed cells. The host cells can be cultured on a stationary substrate or in suspension. Methods of tissue culture are well known to the skilled artisan (see, e.g., Pollard et al., METHODS IN MOLECULAR BIOLOGY VOL. 5: ANIMAL CELL CULTURE, Humana Press (1990); Masters, ANIMAL CELL CULTURE—A PRACTICAL APPROACH, 3d ed., Oxford University Press (2000)). Furthermore, methods of purifying CAB-2 protein from tissue culture supernants are well established in the art (U.S. Pat. Nos. 5,679,546 and 5,851,528, incorporated herein by reference). In general, standard chromatographic procedures can be employed to purify CAB-2 protein. A preferred method of purification from cell culture supernatant is cation exchange chromatography, e.g. on a Super-Q resin (Tosoh Bioscience, Montgomeryville, Pa.), pH 7.0, elution with 0.25 M NaCl, followed by anion exchange chromatography, e.g. on a SP SEPHAROSE™ XL resin (GE Healthcare Life Sciences, Piscataway, N.J.), pH 4.0, elution with 0.15 M NaCl, followed by a second cation exchange chromatography, pH 8.0, elution with 0.15 M NaCl, then a diafiltration step to concentrate CAB-2 protein and exchange the buffer to suit the desired use, e.g. drug formulation, of the protein. Significantly, the invention will also facilitate purification of the CAB-2 protein or a variant thereof because of the higher level of protein production achieved by the inventive nucleic acid molecules.

The nucleic acid molecules of the invention provide a CAB-2 protein which is an inhibitor of complement activation. Methods to analyze the activity of CAB-2 are described, e.g. in U.S. Pat. Nos. 5,679,546 and 5,851,528. The sheep red blood cell lysis assay (Rose et al. (1986) Manual of Clinical Laboratory Immunology, American Society of Microbiology) is one useful method for comparison of the activity of CAB-2 produced from the initial nucleic acid (SEQ ID NO:1) with the activity of CAB-2 produced by a nucleic acid with mutations to increase the amount of full length transcript. In a preferred embodiment, the complement inhibitory activities of the CAB-2 proteins produced from expression of the different nucleic acids are substantially equivalent, for expression in a given cell type under identical conditions.

Uses for CAB-2 also are described, e.g. in U.S. Pat. Nos. 5,679,546 and 5,851,528. Briefly, CAB-2 protein can be administered in the treatment of disorders associated with inordinate and/or excessive activation of the complement system. The administration of CAB-2 in vivo will enable the protein to bind endogenous C3/C5 convertases and inhibit the generation of additional C3b and C5b, of C3a and C5a anaphylatoxins, and of C5b-9 lytic complexes. By inhibition of the generation of anaphylotoxins, CAB-2 can be used to inhibit neutrophil recruitment to sites of inflammation.

An effective amount of CAB-2 is administered to an individual in order to treat such a disease. For example, CAB-2 protein can be used to treat tissue damage due to ischemia-reperfusion following myocardial infarction, aneurysm, cerebral infarction (stroke), hemorrhagic shock, cardiopulmonary bypass surgery, thromboembolism or crush injury; acute inflammation from burns, endotoxemia, septic shock and adult respiratory distress syndrome (ARDS); hyperacute rejection of grafts; inflammatory disorders such as Crohn's disease, sickle cell anemia, asthma, dermatitis, psoriasis and pancreatitis. Autoimmune disorders including, but not limited to, systemic lupus erythematosis, rheumatoid arthritis, glomerulonephritis, macular degeneration, and multiple sclerosis, can also be treated with CAB-2 protein.

For therapy, an effective amount will be sufficient to achieve the desired therapeutic (including prophylactic) effect (such as an amount sufficient to inhibit complement activation). CAB-2 can be administered in a unit dose or multiple doses. For example, the dose is administered at about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. CAB-2 can be administered alone or in combination with another agent. An additional active ingredient (e.g., an anti-inflammatory compound, e.g., steroidal or non-steroidal) can be administered with CAB-2. A variety of routes of administration can be used, as known in the art. Formulation will vary according to the route of administration, as is known in the art.

The following examples further illustrate the invention but, of course, should not be construed as in any way limiting its scope.

### EXAMPLE 1

This example demonstrates that the low level of production of CAB-2 protein can be associated with the production of nonproductive transcripts by the nucleic acid molecule encoding the CAB-2 protein. Specifically, premature polyadenylation sites and cryptic splice sites were identified in the CAB-2 nucleic acid molecule and found to be responsible, at least in part, for the low level of production of the CAB-2 protein.

A CAB-2 cDNA encoding amino acids 1-288 of MCP and 1-322 of DAF in tandem was amplified by PCR from a previously-constructed vector using primers that incorporated unique restriction sites at the 5′ and 3′ ends. The amplified cDNA was cloned into a mammalian expression vector containing a functional promoter and a mouse immunoglobulin kappa light chain 3′ untranslated region along with selective marker genes, such as the gene encoding resistance to G418. Linearized vectors were transfected into CHO-K1 cells by electroporation or with linear polyethylenamine (Polysciences, Inc, Warrington, Pa.). Following recovery, transfected cells were seeded in 96-well plates so as to yield one colony per well.

For transfection, CHO-K1 cells were maintained in Ex-Cell 301 medium (Biosciences, Lenexa, Kans.). Transfectants were selected and propagated in Ex-Cell 301 or Ex-Cell 302 medium containing 8 g/L G418 (Invitrogen, Carlsbad, Calif.) and/or 8 mM histidinol (Sigma Chemicals, St Louis, Mo.). Culture supernatants from CAB-2-protein-expressing clones were evaluated by ELISA at the 96-well and 24-well stages. The highest-producing clones were further evaluated with a shake flask test performed in selection-free media at a starting cell density of 2×10⁵ cells/ml. Culture supernatants were assayed for CAB-2 protein on Day 13 or 14 by ELISA and reverse-phase HPLC.

ELISA plates were pre-coated with a murine monoclonal IgG₁ against MCP. After blocking with PBS containing 1% BSA and 0.5% Tween 20, plates were incubated with cell culture supernatant. This was followed by a two-step incubation with a polyclonal rabbit anti-CAB-2 antibody followed by a mouse anti-rabbit antibody conjugated to horse radish peroxidase. The plates were developed using the TMB detection system (KPL, Gaithersburg Md.) and read on an ELISA plate reader at 450 nm.

CAB-2 protein concentration was further measured using Reverse Phase HPLC (RP-HPLC). Supernatents from extinct cultures were passed through 0.2 micron filters and analyzed using a Poroshell 300SB-C18 column of 2.1×75 mm (Agilent Technologies, Palo Alto, Calif.) with a gradient elution. Protein titer was determined by interpolation of sample peak area with a five point calibration curve ranging from 10 μg/mL to 500 μg/mL.

CAB-2 protein expression was unexpectedly low. To understand the cause for the lower than expected expression levels, CAB-2-specific mRNA, isolated from CHO-K1 transfectants, was analyzed by Northern blot using a cDNA probe encoding the 5′ untranslated region through SCR1 of MCP. Total RNA was isolated from cell lines using TRI-reagent (Molecular Research, Cincinnati, Ohio). RNA from CAB-2-producing clones (20 μg each) was run on an agarose-formaldehyde gel, blotted and hybridized with ³²P-labeled cDNA probes using the Northern Max Kit (Ambion Inc, Austin, Tex.). The integrity and uniformity of the RNA was confirmed by stripping blots and re-hybridizing with a probe for glyceraldehyde phosphate dehydrogenase (GAPDH) as an internal expression control.

Surprisingly, three RNA species were detected, including the expected full length transcript of 2.3 kilobases (Kb) and two transcripts of 1.5 Kb and 790 bases. The 790 base transcript accounted for approximately 50% of CAB-2 mRNA, while the 1.5 Kb RNA accounted for about 10% of the CAB-2 mRNA. When this same blot was hybridized with a probe in the MCP-DAF fusion region of CAB-2, only the 2.3 Kb and 1.5 Kb RNAs were detected while a probe in the DAF STP 3-4 region detected only the full length 2.3 Kb mRNA.

Inspection of the CAB-2 cDNA coding sequence revealed that it contains three copies of the polyadenylation signal, AATAAA (FIG. 2 wherein MCP, sequences from MCP gene; DAF, sequences from the DAF gene; P1-P5, PCR and RACE primers; SP, signal peptide; SCR, short consensus repeats; STP, Serine-Threonine-Proline rich region; TGA, stop codon; bp, base pairs from transcriptional start site). One of these AATAAA sites lies within MCP-derived sequences, 790 base pairs (bp) downstream of the transcription start site, at the sequences encoding amino acids 167-169. The other two AATAAA sites are within DAF-derived sequences, 1811 bp (encoding amino acids 508-509) and 2087 bp (encoding amino acids 600-601) downstream of the CAB-2 transcription start site, respectively. To investigate the use of these putative polyadenylation sites, 3′ RACE was performed.

3′ RACE was performed using a 3′ RACE kit (Invitrogen Corp., Carlsbad, Calif.). Total RNA isolated from CAB-2-transfected cells, cell lines, and various tissues was reverse-transcribed into cDNA using an Adapter (AP) primer. PCR was performed using an Abridged Universal Adapter Primer (AUAP) and two gene-specific forward primers (P4 and P5) located within MCP and DAF (FIG. 2) to amplify any prematurely polyadenylated transcripts. The resulting PCR products were verified by DNA sequence analysis.

3′ RACE using forward primer P4, located in the 5′ untranslated region, 522 base pairs upstream of the AATAAA sequence within MCP (FIG. 2), revealed an about 540 base pair PCR product in all CAB-2-producing transfectants which would result from polyadenylation at this AATAAA site. DNA sequence analysis of this PCR product revealed that it terminates shortly after the internal MCP-derived AATAAA sequence. Polyadenylation at this site would yield the about 790 base pair transcript observed by Northern analysis. 3′ RACE using primer P5, located 342 bp upstream of the 5′ AATAAA site in DAF SCR4, should capture RNA polyadenylated at the two internal DAF-derived AATAAA sites and at the AATAAA site in the 3′ UTR. However, the results demonstrated that only the AATAAA sequence located in DAF SCR4 signals functioned in polyadenylation, resulting in a truncated about 380 base pair PCR product that was confirmed by DNA sequence analysis. As expected, polyadenylation at the AATAAA site in the 3′ UTR yielded an about 890 base pair product.

To investigate whether premature polyadenylation was unique to the chimeric CAB-2 protein or whether it occurs in tissues that express native MCP and DAF proteins, additional RNA sources were studied. 3′ RACE using primer P6 (FIG. 2) located in the MCP signal sequence identified products of a size indicating use of the internal AATAAA site in MCP in salivary gland, peripheral blood leukocytes, placenta, and the human T-cell line, HSB2. DNA sequence analysis of the PCR products from the placenta and HSB2 cells confirmed them to be products from prematurely truncated MCP mRNA. Similarly, using primer P5, 3′ RACE PCR products, indicating use of the internal AATAAA site in DAF SCR4, were observed in the salivary gland and peripheral blood leukocytes. However, DNA sequence analysis demonstrated that only the PCR product from the salivary gland resulted from prematurely polyadenylated DAF mRNA.

RT-PCR was used to identify the source of the 1.5 Kb transcript. Total RNA, isolated as described above, was treated with DNaseI to remove plasmid cDNA. Random hexamer (1 μg) and Oligo dT (0.5 μg) primers were used for first-strand cDNA synthesis using Superscript 11 reverse transcriptase (Invitrogen Corp., Carlsbad, Calif.) and 2 μg of each DNase-1-treated RNA. PCR was performed using 2 μg of first-strand cDNA and primers (P1, P2, and P3) in the 5′ and 3′ untranslated regions flanking the CAB-2 cDNA (FIG. 2).

PCR using primer P1, which lies downstream of the transcription start site in the promoter, and primer P2, located 22 base pairs downstream of the stop codon, amplified the expected full length cDNA of 2.0 Kb. PCR with primer P1 and primer P3, which lies downstream of primer P2 and immediately upstream of the polyadenylation signal in the 3′ UTR, however, yielded a predominant band of about 1.5 Kb. The expected 2.3 Kb cDNA was amplified, but to a much lesser extent. A second, slightly higher molecular weight band also was observed which resulted either from incomplete splicing at the 5′ intron (or from amplification of chromosomal DNA in the RNA preparation.) DNA sequence analysis of the 1.5 Kb product revealed that the mRNA was spliced from a non-canonical donor site in SCR2 from DAF to an acceptor site in the 3′UTR of the vector. This spliced RNA, resulting from excision of 660 bases of DAF mRNA, corresponds to the about 1.5 Kb transcript observed on the Northern blot. The location of this splice event explains the absence of the 1.5 Kb RNA on the Northern blot probed with a cDNA in DAF STP 3-4. Splicing initiating from this site in native DAF was not observed in RNA isolated from brain, salivary gland, kidney, spleen, lung, and peripheral blood leukocytes, or from the cell lines HEK293, Minor, A549, and HSB2, indicating that this process is specific to CAB-2 expressed in CHO cells using the initial unmodified CAB-2 nucleic acid molecule.

These results confirmed that the unexpectedly low levels of CAB-2 protein produced in the CHO-K1 cells containing CAB-2 expression cassette were the result of unexpected nonproductive transcripts encoded by the CAB-2 cassette.

### EXAMPLE 2

This example demonstrates that specific changes in a CAB-2 nucleic acid molecule can improve CAB-2 protein production.

The CAB-2 nucleic acid molecule described in Example 1 was modified in an effort to increase the production of CAB-2 protein. In particular, the CAB-2 nucleic acid molecule was modified to eliminate, or at least minimize, the generation of nonproductive transcripts. The two functional polyadenylation sequences and the non-canonical splice donor sequence identified in Example 1 were modified by replacing two nucleotides at each site without changing the native protein encoded by the CAB-2 nucleic acid molecule. The first and second AATAAA sites were changed to AAGATC and AACAAG, respectively. The splice donor site was changed from GAACCTTCT to GAGCCCTCT. Changes in the CAB-2 nucleic acid sequence to correct the two functional, internal AATAAA sites and the splice donor site were made using inverse PCR and DpnI digestion. Complementary primers overlapping the site of change were used to PCR amplify the whole plasmid in a mixture containing 500 mM dNTPs, 2 units of Pfu polymerase, 250 ng each of sense and antisense primers, and 200 ng of CAB-2 plasmid DNA. PCR involved 18 cycles with an extension time of 2.5 minutes for each Kb of DNA. PCR products were treated with DpnI (which only digests the adenine-methylated plasmid DNA) and transformed into Escherichia coli DH5α cells. Transformants were screened by restriction enzyme digestion for incorporation of the changes and then confirmed by DNA sequence analysis.

To evaluate the benefit of these modifications, plasmids with changes at each individual site and at multiple sites were transiently expressed in CHO-K1 cells. RNA isolated from the transient transfectants and tested by RT-PCR and 3′ RACE confirmed that these modifications eliminated the nonproductive transcription products.

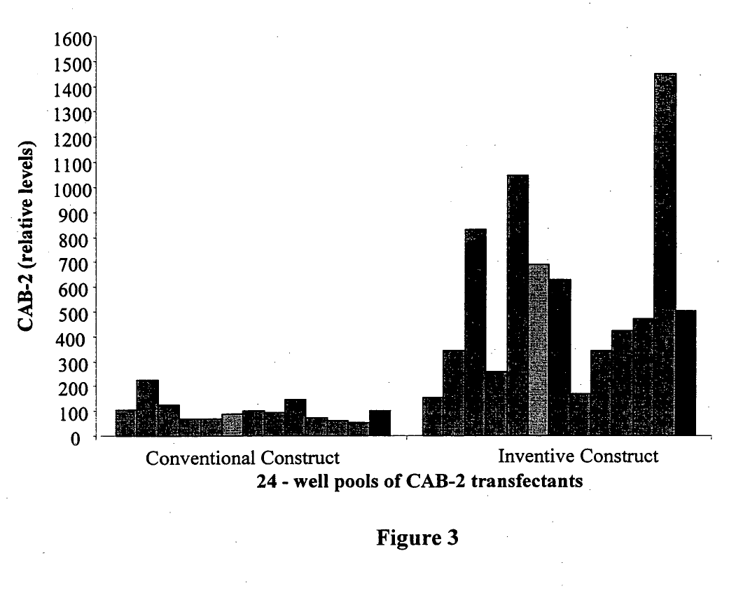

To determine whether these changes improved protein expression levels, CHO-K1 cells were stably transfected (transformed) with a plasmid containing the CAB-2 cDNA modified at all three positions (SEQ ID NO: 2). Expression levels were compared in parallel using CHO-K1 cells that were stably transfected with a plasmid containing the initial unmodified CAB-2 cDNA (SEQ ID NO: 1). Specifically, culture supernatants from extinct pools of stable transfectants were assayed by ELISA to determine the amounts of produced CAB-2 protein for both the initial unmodified CAB-2 nucleic acid molecule and the modified CAB-2 nucleic acid molecule. The results of these comparative tests are set out in FIG. 3. As is apparent from FIG. 3, the results revealed that the modified CAB-2 nucleic acid molecule produced, on average, 5-fold higher CAB-2 titers than transfectants generated with the initial unmodified CAB-2 nucleic acid molecule. Moreover, RNA isolated from individual stable clones generated by transfection with the modified CAB-2 nucleic acid molecule and analyzed by 3′ RACE directed to the MCP-derived and DAF-derived nonproductive transcripts confirmed the absence of products resulting from the prematurely polyadenylated RNA. RT-PCR confirmed that the 1.5 Kb product from alternative splicing also no longer occurred. A Northern blot also confirmed the absence of truncated RNA forms in cell lines developed with the modified CAB-2 nucleic acid molecule, correlating elimination of nonproductive RNAs with increased CAB-2 protein production levels.

These results confirmed that the elimination of premature polyadenylation sites and cryptic splice sites by site-directed mutagenesis results in increased CAB-2 protein production from a cloned CAB-2 gene transformed into and expressed by eukaryotic cells.

### EXAMPLE 3

Studies were conducted to compare the ability of CAB-2 produced by expression of conventional nucleic acid (SEQ ID NO:1) or by a mutated nucleic acid (SEQ ID NO:2) in reducing complement activation caused by exposure of whole blood to an extracorporeal circuit (EC). The EC (Radnoti Liver Perfusion system, Monrovia, Calif.) consisted of a peristaltic pump, membrane oxygenator, reservoir, and plastic and glass tubing, primed with Lactated Ringers priming solution (4.0 g/L dextrose, 4.0 g/L mannitol, 5 U/ml porcine heparin) maintained at 37° C. Phosphate buffered saline or CAB-2 produced from nucleic acid having a sequence of SEQ ID NO:1 or SEQ ID NO:2 was added to human blood to make a blood concentration of 0, 30, or 300 μg/ml CAB-2. Blood was circulated and samples were collected over 90 minutes. Plasma was assayed with ELISA kits for complement activation markers C3a (Quidel Corp., San Diego, Calif.), SC5b-9 (Quidel Corp., San Diego, Calif.), and neutrophil elastase (Milenia Biotec, Bad Nauheim, Del.). FACS analysis was performed to determine monocyte CD11b upregulation. 5 and 90 minute samples were sent for complete blood cell (CBC) count for quantification of platelet numbers, polymorphonuclear (PMN) and monocyte numbers.

All time versus marker curves followed the same general pattern of beginning from a baseline value rising exponentially and finally tapering off to a peak response above baseline. The addition of 30 μg/ml CAB-2 resulted in a 57.3% reduction in C3a response, and an 11.4% decline in SC5b-9 peak response, relative to their respective control peak response. Furthermore, increasing the dose to 300 μg/ml CAB-2 resulted in an 89.0% decrease in C3a peak response, and a 91.7% reduction in SC5b-9 peak response relative to control. No significant difference in the dose-response effect was found between CAB-2 produced by expression of a nucleic acid with a sequence of SEQ ID NO:1 or SEQ ID NO:2. A comparison of the 90 minute time point from CD11b upregulation and neutrophil elastase concentrations did not show any discernable dose or treatment effect. This is believed to be due to excessive contact activation of PMN cells due to the laboratory nature of the components used in this EC model. No significant difference was seen in platelet, neutrophil, or monocyte cell counts between the 5 and 90 minute time points from the CBC data, thus there was no indication of cell sticking to the components of EC. Results are detailed in Table 1 below.

TABLE 1
Comparison of activity of CAB-2 produced from expression of
conventional (SEQ ID NO:1) versus mutant (SEQ ID NO:2) nucleic acid
sequence. Measurements of ng/ml levels of C3a, SC5b-9, PMN Elastase
and CD11b at 90 min.

| | | | | CD11b |
|---|---|---|---|---|
| Study | C3a | SC5b-9 | PMN Elastase | upregulation |
| Control | 170 ± 23 | 202 ± 24 | 2626 ± 1302 | 118 ± 6 |
| (0 min) | n = 5 | n = 5 | n = 5 | n = 5 |
| Control | 3579 ± 948 | 2406 ± 312 | 10888 ± 4415 | 390 ± 35 |
| (90 min) | n = 5 | n = 5 | n = 5 | n = 5 |
| 30 μg/ml | 1988 ± 384 | 1520 ± 335 | 30168 ± 15923 | 340 ± 36 |
| from | n = 4 | n = 4 | n = 4 | n = 4 |
| conventional | | | | |
| 300 μg/ml | 410 ± 60 | 389 ± 22 | 8038 ± 1168 | 470 ± 72 |
| from | n = 4 | n = 4 | n = 4 | n = 4 |
| conventional | | | | |
| 30 μg/ml | 1220 ± 214 | 1192 ± 204 | 15257 ± 5970 | 320 ± 10 |
| from mutant | n = 4 | n = 4 | n = 4 | n = 4 |
| 300 μg/ml | 341 ± 45 | 324 ± 55 | 11509 ± 6003 | 367 ± 158 |
| from mutant | n = 4 | n = 4 | n = 4 | n = 4 |

All references, including publications, patent applications, and patents, cited herein are hereby incorporated by reference to the same extent as if each reference were individually and specifically indicated to be incorporated by reference and were set forth in its entirety herein. The contents of the Figures and the Sequence Listing, also are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A nucleic acid molecule consisting of a mutagenized sequence of SEQ ID NO: 1, wherein the mutation is selected from the group consisting of A (501)→G, A (504)→C or T, A (1170)→G, T (1173)→C, A, or G, T (1524)→C, A (1527)→G, T (1800)→C, and A (1803)→G, and combinations thereof.

2. The nucleic acid molecule of claim 1, wherein there are at least two mutations and wherein
(a) at least one mutation is selected from the group consisting of A (501)→G, A (504)→C or T, T (1524)→C, A (1527)→G, T (1800)→C, and A (1803)→G, and
(b) at least one mutation is selected from the group consisting of A (1170)→G, and T (1173)→C, A, or G.

3. The nucleic acid molecule of claim 1, wherein the nucleic acid molecule is selected from the group consisting of a nucleic acid with the sequence of SEQ ID NO: 2 and a nucleic acid with the nucleotide sequence of the DNA insert of the plasmid deposited with ATCC as Accession Number ______.

4. A nucleic acid molecule encoding a protein of the amino acid sequence SEQ ID NO: 3, wherein expression of the nucleic acid molecule produces protein of SEQ ID NO: 3 at a level of at least 60% that of a nucleic acid molecule consisting of the sequence of SEQ ID NO: 2 or the nucleotide sequence of the DNA insert of the plasmid deposited with ATCC as Accession Number ______ when transformed into CHO-K1 cells under identical conditions.

5. A construct comprising control elements and the nucleic acid molecule of claim 1 operatively linked thereto for expression of the nucleic acid molecule to produce a protein of SEQ ID NO: 3.

6. A construct comprising control elements and the nucleic acid molecule of claim 2 operatively linked thereto for expression of the nucleic acid molecule to produce a protein of SEQ ID NO: 3.

7. A construct comprising control elements and the nucleic acid molecule of claim 3 operatively linked thereto for expression of the nucleic acid molecule to produce a protein of SEQ ID NO: 3.

8. A eukaryotic cell transformed with a nucleic acid molecule of claim 1.

9. The eukaryotic cell of claim 8, wherein the cell is a CHO-K1 cell.

10. A eukaryotic cell transformed with a nucleic acid molecule of claim 2.

11. The eukaryotic cell of claim 10, wherein the cell is a CHO-K1 cell.

12. A eukaryotic cell transformed with a nucleic acid molecule of claim 3.

13. The eukaryotic cell of claim 12, wherein the cell is a CHO-K1 cell.

14. A eukaryotic cell transformed with a nucleic acid molecule of claim 4.

15. The eukaryotic cell of claim 14, wherein the cell is a CHO-K1 cell.

16. A method of producing CAB-2 protein comprising:
(a) providing cells transformed with a nucleic acid molecule of claim 1, and
(b) culturing the transformed cells to produce CAB-2 protein.

17. A method of producing CAB-2 protein comprising:
(a) providing cells transformed with a nucleic acid molecule of claim 2, and
(b) culturing the transformed cells to produce CAB-2 protein.

18. A method of producing CAB-2 protein comprising:
(a) providing cells transformed with a nucleic acid molecule of claim 3, and
(b) culturing the transformed cells to produce CAB-2 protein.

19. A method of producing CAB-2 protein comprising:
(a) providing cells transformed with a nucleic acid molecule of claim 4, and
(b) culturing the transformed cells to produce CAB-2 protein.

20. The nucleic acid molecule of claim 4 wherein the nucleic acid has at least one base mutation in a site selected from the group consisting of:
(a) bases 501 to 506 of SEQ ID NO:1;
(b) bases 1170 to 1173 of SEQ ID NO:1;
(c) bases 1522 to 1527 of SEQ ID NO:1;
(d) bases 1665 to 1670 of SEQ ID NO:1; and
(e) bases 1798 to 1803 of SEQ ID NO:1.


SEQUENCE LISTING (4 sequences)

SEQ ID NO: 1 - DNA, 1836 bp, Artificial
  Synthetic
atggagcctcccggccgccgcgagtgtccctttccttcctggcgctttcctgggttgcttctggcggccatggtgttgctgctgtactccttctccgatgcctgtgaggagccaccaacatttgaagctatggagctcatcgggaaaccaaaaccctactatgagattggtgaacgagtagattataagtgtaaaaaaggatacttctatatacctcctcttgccacccacactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggaaaaccccccatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgtcgactgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattacttccctgtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtctaataagcggctcgagtgtccagtggagtgacccgttgccagagtgcagagaaatttattgcccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgttga

SEQ ID NO: 2 - DNA, 1836 bp, Artificial
  Synthetic
atggagcctcccggccgccgcgagtgtccctttccttcctggcgctttcctgggttgcttctggcggccatggtgttgctgctgtactccttctccgatgcctgtgaggagccaccaacatttgaagctatggagctcatcgggaaaccaaaaccctactatgagattggtgaacgagtagattataagtgtaaaaaaggatacttctatatacctcctcttgccacccacactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggaaaaccccccatatgtgaaaaggttttgtgtacaccacctccaaagatcaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgtcgactgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattacttccctgtcggtactgttgtggaatatgagtgccgtccaggttacagaagagagccctctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtctaataagcggctcgagtgtccagtggagtgacccgttgccagagtgcagagaaatttattgcccagcaccaccacaaattgacaatggaataattcaaggggaacgtgaccattatggatatagacagtctgtaacgtatgcatgtaacaagggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgttga

SEQ ID NO: 3 - protein, 611 aa, Artificial
  Synthetic
MEPPGRRECPFPSWRFPGLLLAAMVLLLYSFSDACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVSTDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTR

SEQ ID NO: 4 - DNA, 1836 bp, Artificial
  Synthetic
atggagcctcccggccgccgcgagtgtccctttccttcctggcgctttcctgggttgcttctggcggccatggtgttgctgctgtactccttctccgatgcctgtgaggagccaccaacatttgaagctatggagctcatcgggaaaccaaaaccctactatgagattggtgaacgagtagattataagtgtaaaaaaggatacttctatatacctcctcttgccacccacactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggaaaaccccccatatgtgaaaaggttttgtgtacaccacctccaaagatyaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgtcgactgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattacttccctgtcggtactgttgtggaatatgagtgccgtccaggttacagaagagagccvtctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtctaataagcggctcgagtgtccagtggagtgacccgttgccagagtgcagagaaatttattgcccagcaccaccacaaattgacaatggaataattcaaggggaacgtgaccattatggatatagacagtctgtaacgtatgcatgtaacaagggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaacaagggaagtggaaccacttcaggtactacccgttga